(19) United States
(12) Patent Application Publication
(10) Pub. No.: US 2023/0218880 A1
(43) Pub. Date: Jul. 13, 2023

(54) Integrated Catheter System with Near Patient Access Port

(71) Applicant: Becton, Dickinson and Company, Franklin Lakes, NJ (US)

(72) Inventor: Jonathan Karl Burkholz, Salt Lake City, UT (US)

(21) Appl. No.: 18/094,688

(22) Filed: Jan. 9, 2023

Related U.S. Application Data
(60) Provisional application No. 63/298,032, filed on Jan. 10, 2022.

(51) Int. Cl. A61M 39/16; A61M 39/10; A61M 39/22

(52) U.S. Cl. CPC A61M 39/162; A61M 39/105; A61M 39/22

(57) ABSTRACT

An integrated catheter system includes a catheter adapter including a catheter and an inlet, the catheter configured to be inserted into a patient's vasculature, a needle-free connector including a first port, a second port positioned opposite the first port, and a side port positioned between the first port and the second port, the second port including a valve member, intermediate tubing extending between the inlet of the catheter adapter and the first port of the needle-free connector, and extension tubing extending from the side port of the needle-free connector, where the needle-free connector includes chlorhexidine.

### CROSS-REFERENCE TO RELATED APPLICATION

The present application claims priority to U.S. Provisional Application Ser. No. 63/298,032, entitled “Integrated Catheter System with Near Patient Access Port”, filed Jan. 10, 2022, the entire disclosure of which is hereby incorporated by reference in its' entirety.

### BACKGROUND OF THE INVENTION

### Field of the Invention

The present disclosure relates to an integrated catheter system with a near patient access port.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of a blood draw device is shown and described in U.S. Pat. No. 11,090,461, which is hereby incorporated by reference in its entirety.

### SUMMARY OF THE INVENTION

In one aspect or embodiment, an integrated catheter system includes a catheter adapter including a catheter and an inlet, the catheter configured to be inserted into a patient's vasculature, a needle-free connector including a first port, a second port positioned opposite the first port, and a side port positioned between the first port and the second port, the second port including a valve member, intermediate tubing extending between the inlet of the catheter adapter and the first port of the needle-free connector, and extension tubing extending from the side port of the needle-free connector, where the needle-free connector includes chlorhexidine.

The chlorhexidine may be at least one of chlorhexidine diacetate and chlorhexidine gluconate. The needle-free connector may include a lube comprising the chlorhexidine. The needle-free connector may include a coating including the chlorhexidine. The needle-free connector may include an insert received within the needle-free connector, with the insert including the chlorhexidine. The needle-free connector may include a flow diverting feature, with the flow diverting feature including an insert or coating comprising the chlorhexidine.

The needle-free connector may include a body defining a longitudinal axis extending between the first port and the second port, where the side port extends from the body at an angle of 30-150 degrees relative to the longitudinal axis of the body. The side port may be in fluid communication with the body of the needle-free connector via an inlet, where the inlet is offset from the longitudinal axis of the body of the needle-free connector. The flow diverting feature may include a lead-in configured to guide movement of a probe through the first port and/or second port. The lead-in may include a frusto-conical surface. The chlorhexidine may be configured to elute over a predetermined time period.

In accordance with an embodiment of the present invention, a catheter system includes a catheter adapter including a catheter and an inlet, the catheter configured to be inserted into a patient's vasculature, and a needle-free connector including a first port, a second port positioned opposite the first port, and a side port positioned between the first port and the second port, the second port comprising a valve member, wherein the needle-free connector includes chlorhexidine.

In accordance with an embodiment of the present invention, the chlorhexidine includes at least one of chlorhexidine diacetate and chlorhexidine gluconate.

In accordance with an embodiment of the present invention, the needle-free connector includes a lube comprising the chlorhexidine.

In accordance with an embodiment of the present invention, the needle-free connector comprises a coating comprising the chlorhexidine.

In accordance with an embodiment of the present invention, the needle-free connector comprises an insert received within the needle-free connector, the insert comprising the chlorhexidine.

In accordance with an embodiment of the present invention, the needle-free connector comprises a flow diverting feature, the flow diverting feature comprising an insert or coating comprising the chlorhexidine.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an integrated catheter system according to one aspect or embodiment of the present application;

FIG. 2 is a cross-sectional view of a needle-free connector of the system of FIG. 1 according to one aspect or embodiment of the present application;

FIG. 3 is a cross-sectional view of a needle-free connector of the system of FIG. 1 according to a further aspect or embodiment of the present application; and

FIG. 4 is a cross-sectional view of a needle-free connector of the system of FIG. 1 according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

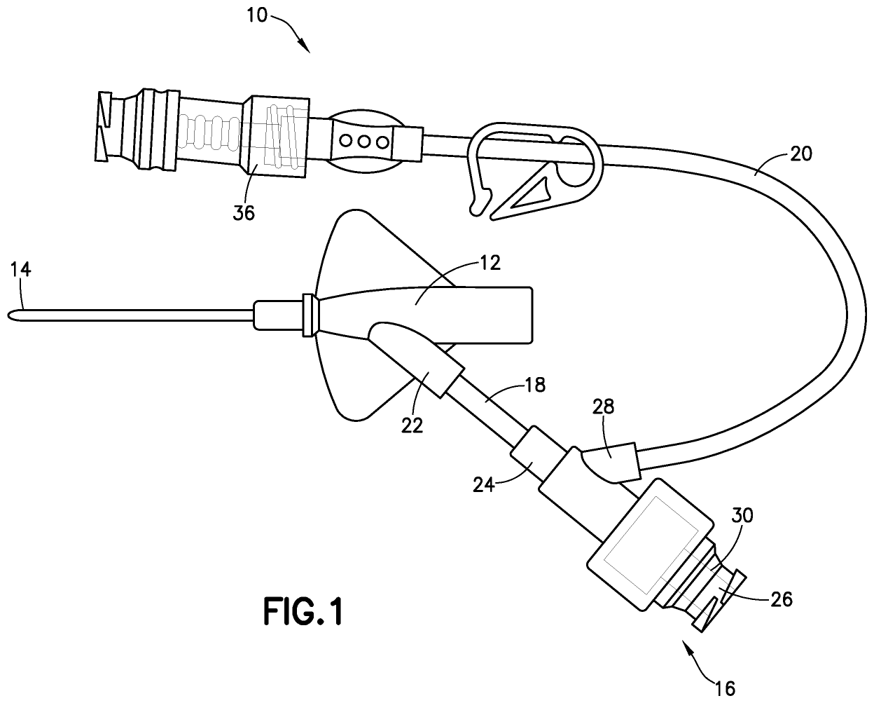

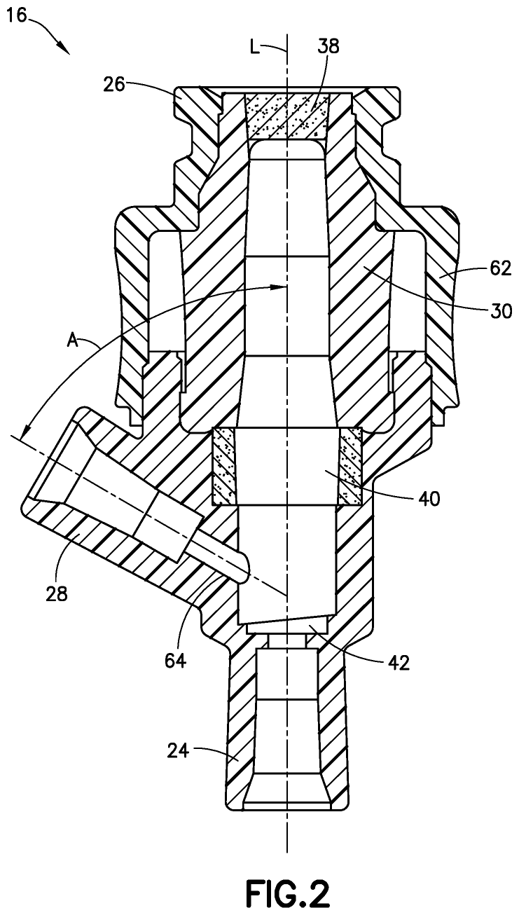

Referring to FIGS. 1 and 2, an integrated catheter system 10 includes a catheter adapter 12 having a catheter 14 configured to be inserted into a patient's vasculature, a needle-free connector 16, intermediate tubing 18, and extension tubing 20. The catheter adapter 12 includes an inlet 22. The needle-free connector 16 includes a first port 24, a second port 26 positioned opposite the first port 24, and a side port 28 positioned between the first port 24 and the second port 26. The second port 26 includes a valve member 30. The intermediate tubing 18 extends between the inlet 22 of the catheter adapter 12 and the first port 24 of the needle-free connector 16. The extension tubing 20 extends from the side port 28 of the needle-free connector 16. The intermediate tubing 18 is configured to provide flexibility when inserting and dressing the catheter 14 and also when manipulating the needle-free connector 16 for flushing, blood draw, and/or other procedure without disturbing the catheter insertion site. The proximity of the needle-free connector 16 to the catheter adapter 12 is configured to enable a blood draw device 32 to protrude a sufficient distance beyond an end of the catheter 14. The extension tubing 20 is configured to be utilized for introducing of intravenous fluid, medication, etc.

In one aspect or embodiment, the intermediate tubing 18 has a length of 6-8 mm. In a further aspect or embodiment, the intermediate tubing 18 has a maximum length of 15 mm. A longer length of the intermediate tubing 18 improves insertion ergonomics, flexibility of access and securement, and ease of dressing. However, increasing the length of the intermediate tubing 18 decreases the distance the blood draw device 32 will be able to protrude from the catheter 14 unless the blood draw device 32 allows for a longer tubing. In one aspect or embodiment, the intermediate tubing 18 has a length that is shorter than a length of the extension tubing 20. The intermediate tubing 18 may have a length configured to allow a probe of the blood draw device 32 extend a predetermined length beyond a tip of the catheter 14.

Referring to FIG. 1, in one aspect or embodiment, the integrated catheter system 10 includes a needle hub assembly (not shown) and a medical component 36, such as luer port connector. The needle hub assembly is assembled with the catheter adapter 12 by inserting a needle (not shown) into a lumen of the catheter 14. In one aspect or embodiment, the needle hub assembly includes a needle shield configured to secure a tip of the needle within the needle shield after use. The needle shield may be activated passively. In one aspect or embodiment, the catheter adapter 12 includes one or more wings, as shown, configured to engage a skin surface of a patient. In another aspect or embodiment, the catheter adapter 12 does not include wings.

When a blood draw device (not shown) is connected to the second port 26 of the needle-free connector 16, a flow tube of the blood draw device can be extended through the needle-free connector 16, through the intermediate tubing 18, the inlet 22 of the catheter adapter 12, and the catheter 14. The blood draw device may be the PIVO™ blood draw device commercially available from Velano Vascular. In one aspect or embodiment, the blood draw device is the same or similar to the blood draw device shown in U.S. Pat. No. 11,090,461, which is hereby incorporated by reference in its entirety. In one aspect or embodiment, the blood draw device may be any device that advances tubing, a probe, a guidewire, instrument, and/or sensor into the fluid path of the integrated catheter system 10 or beyond the tip of the catheter 14. In one aspect or embodiment, the needle-free connector 16 is similar and operates similarly to the connector shown and described in U.S. Patent Application Publication No. 2019/0160275, which is hereby incorporated by reference in its entirety.

Referring to FIGS. 1 and 2, in one aspect or embodiment, the integrated catheter system 10 is optimized for compatibility with the blood draw device and for reducing the risk of catheter access associated injection. The system 10 includes the needle-free connector 16 that includes optimized flush-ability features, integrated antimicrobial features, optimized line draw device or probe guiding fluid path, remote luer connection to reduce side manipulation, catheter stabilization platform, and/or a direct probe access fluid path to allow a probe to be advanced into the catheter fluid path and beyond the tip of the catheter.

Referring to FIG. 2, in one aspect or embodiment, the needle-free connector 16 includes chlorhexidine. In one aspect or embodiment, the chlorhexidine includes at least one of chlorhexidine diacetate and chlorhexidine gluconate. The chlorhexidine diacetate and chlorhexidine gluconate are configured to be provided with an optimal concentration and elution profile for a safe and effective antimicrobial activity profile. As shown in FIG. 2, the needle-free connector 16 includes a lube 38 containing the chlorhexidine. The lube 38 may be positioned on the valve member 30 of the needle-free valve 16, such as on a slit of the valve member 30, although other suitable locations may be utilized. The lube 38 may be a silicon lubricant. In further aspects or embodiments, the needle-free connector 16 includes a coating or insert 40 received within the needle-free connector 16, with the coating and/or insert 40 containing the chlorhexidine. The insert 40 can be formed with a geometry configured to allow the insert 40 to be compression fit within the needle-free connector 16 to prevent the insert 40 from moving or becoming dislodged once inserted into the connector 16. The material of the insert 40 may be hygroscopic so that the insert 40 swells when subject to a fluid thereby enhancing the compression fit of the insert 40 within the connector 16. The material of the insert 40 may be reinforced using an internal structure. The insert 40 may be formed by casting, thermoforming, or extrusion. The chlorhexidine may be included within a hydroscopic UV cured adhesive coating, insertion, and/or lubricant. Other antimicrobial agents may include silver or other additives within a coating or within the molded components.

The needle-free connector 16 includes a flow diverting feature 42 configured to improve the flush-ability of the needle-free connector 16, such as creating turbulent flow, swirling, and/or vortex within the connector 16. In one aspect or embodiment, the chlorhexidine is configured to elute over a predetermined period of time, such as the dwell time of the catheter 14. The chlorhexidine may be configured to elute when subjected to a fluid within the needle-free connector 16. The chlorhexidine is configured to be antimicrobial and kill microbes that may be introduced to or present in the fluid path of the system 10.

Suitable anti-microbial lubricants are described in U.S. Pat. Nos. 9,956,379; 9,675,793; 8,426,348; and 8,754,020; 8,691,881, which are each hereby incorporated by reference in their entirety. Suitable anti-microbial inserts or coating technology, including the anti-microbial agent concentration, matrix volume, surface area, and elution profile, is described in U.S. Pat. Nos. 10,792,399; 10,792,398; and 10,589,063, which are each hereby incorporated by reference in their entirety. Examples of suitable formulations and methods that can be used to apply the formulations on the needle-free connector are described in U.S. Patent Application Publication No. 2014/0200523 titled “Systems and Methods for Applying a Novel Antimicrobial Coating Material to a Medical Device”, which is hereby incorporated by reference in its entirety.

In certain aspects and embodiments, an antimicrobial coating can be formed of a matrix that includes one or more antimicrobial agents. For example, the matrix can be a polymer or other suitable material that is cured (e.g. via UV curing) or otherwise bonded to the surface of the connector. In such coatings, the antimicrobial agent will be eluted from the matrix coating in a controlled fashion. Examples of suitable matrixes and methods of applying the matrixes that can be employed to form an antimicrobial coating on a connector are described in U.S. Pat. No. 8,512,294, titled “Vascular Access Device Antimicrobial Materials and Solutions”; U.S.pPatent application Ser. No. 12/397,760, titled “Antimicrobial Compositions”; U.S. application Ser. No. 12/476,997, titled “Antimicrobial Coating Compositions”; U.S. application Ser. No. 12/490,235, titled “Systems and Methods for Applying an Antimicrobial Coating to a Medical Device”; and U.S. application Ser. No. 12/831,880, titled “Antimicrobial Coating for Dermally Invasive Devices”, which are each hereby incorporated by reference in their entirety.

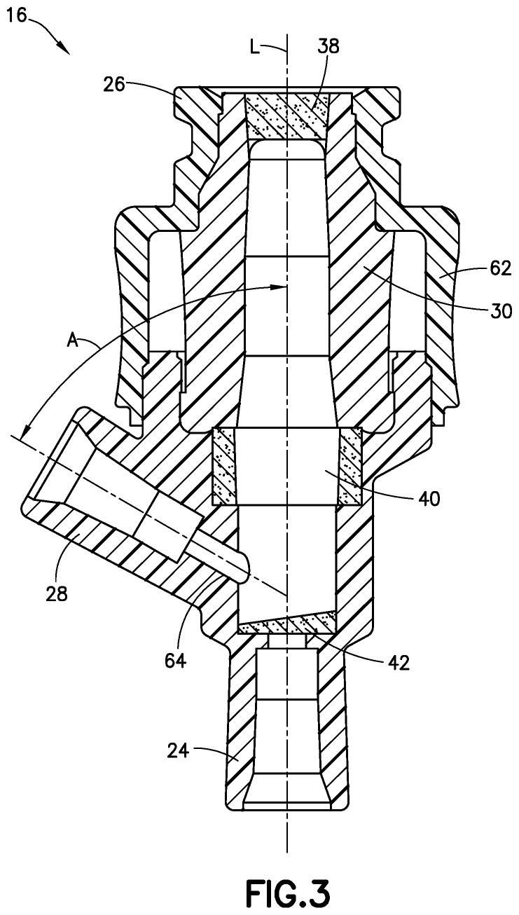

Referring to FIG. 3, in one aspect or embodiment, the flow diverting feature 42 includes an insert or coating containing the chlorhexidine.

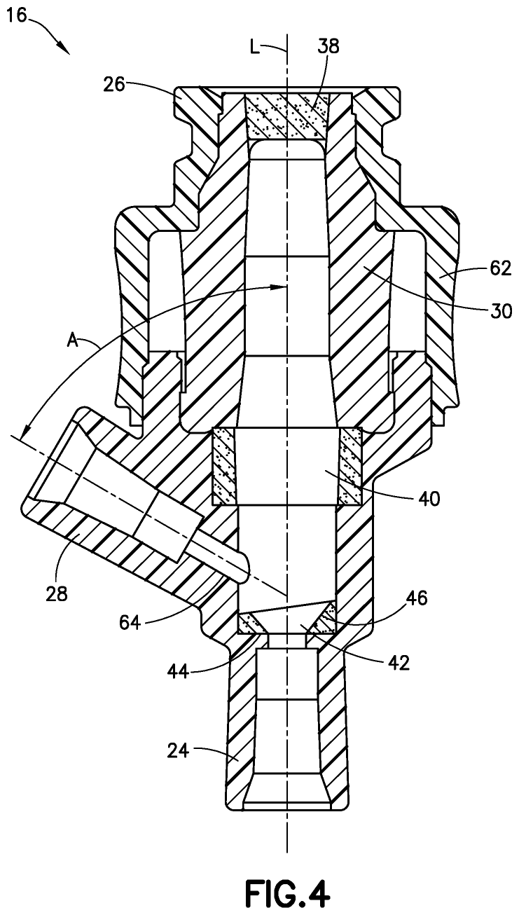

Referring to FIG. 4, in one aspect or embodiment, the flow diverting feature 42 includes a lead-in 44 configured to guide movement of a probe through the first port 24 and/or second port 26 of the needle-free connector 16. The lead-in 44 includes a frusto-conical surface 46, although other suitable structures or features may be utilized.

Referring to FIGS. 2-4, a body 62 of the needle-free connector 16 defines a longitudinal axis L extending between the first port 24 and the second port 26, with the side port 28 extending from the body 62 at an angle A of 30-150 degrees relative to the longitudinal axis L of the body 62. In one aspect or embodiment, the side port 28 extends from the body 62 at an angle of 60 degrees relative to the longitudinal axis L of the body 62. The side port 28 extends at the angle A toward the second port 26, although other suitable arrangements may be utilized. The side port 28 is in fluid communication with the body 62 of the needle-free connector 16 via an inlet 64, where the inlet 64 is offset from the longitudinal axis L of the body 62 of the needle-free connector 16. The angle A and the offset of the side port 28 is configured to improve the flush-ability of the needle-free connector 16 by creating turbulent flow, swirling, and/or vortex within the connector 16.

In one aspect or embodiment, at least a portion of the needle-free connector 16 is transparent. The connector components of the integrated catheter 10 may be transparent, opaque, and/or colored. In one aspect or embodiment, the needle-free connector 16 includes an anti-reflux valve.

In one aspect or embodiment, the medical component 36 at the end of the extension tubing 20 is a single port or dual port connector and may include a variety of connectors, including needle-free connectors or needle access connectors, such as a PRN. The extension tubing 20 may be left or right facing. In one aspect or embodiment, in addition to a vent plug, the medical component 36 may be a removable or non-removable needle free connector or needle access connectors, such as a PRN, that is attached to a female luer connection provided on the extension tubing 20. In one aspect or embodiment, a dual female luer port may be bonded or otherwise attached to the extension tubing 20 instead of a single luer connector.

In one aspect or embodiment, the needle-free connector 16 is configured to be flushed via a single flushing process via the side port 28.

The system 10 incorporates flush-ability and antimicrobial features to synergistically improve catheter performance by reducing the risk of device contamination and patient infection by combining two critical tools to reduce infection risk. Reduce the number of microbes in a system (improved flushing) and decreasing the remaining microbe's ability to survive (antimicrobials). These benefits, in combination with the other integrated catheter benefits, may reduce the overall risk of catheter complications and provide integrated catheter probe access.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An integrated catheter system comprising:
a catheter adapter comprising a catheter and an inlet, the catheter configured to be inserted into a patient's vasculature;
a needle-free connector comprising a first port, a second port positioned opposite the first port, and a side port positioned between the first port and the second port, the second port comprising a valve member;
intermediate tubing extending between the inlet of the catheter adapter and the first port of the needle-free connector; and
extension tubing extending from the side port of the needle-free connector,
wherein the needle-free connector comprises chlorhexidine.

2. The system of claim 1, wherein the chlorhexidine comprises at least one of chlorhexidine diacetate and chlorhexidine gluconate.

3. The system of claim 1, wherein the needle-free connector comprises a lube comprising the chlorhexidine.

4. The system of claim 1, wherein the needle-free connector comprises a coating comprising the chlorhexidine.

5. The system of claim 1, wherein the needle-free connector comprises an insert received within the needle-free connector, the insert comprising the chlorhexidine.

6. The system of claim 1, wherein the needle-free connector comprises a flow diverting feature, the flow diverting feature comprising an insert or coating comprising the chlorhexidine.

7. The system of claim 1, wherein the needle-free connector comprises a body defining a longitudinal axis extending between the first port and the second port, and wherein the side port extends from the body at an angle of 30-150 degrees relative to the longitudinal axis of the body.

8. The system of claim 1, wherein the side port is in fluid communication with the body of the needle-free connector via an inlet, and wherein the inlet is offset from the longitudinal axis of the body of the needle-free connector.

9. The system of claim 6, wherein the flow diverting feature comprises a lead-in configured to guide movement of a probe through the first port and/or second port.

10. The system of claim 9, wherein the lead-in comprises a frusto-conical surface.

11. The system of claim 1, wherein the chlorhexidine is configured to elute over a predetermined time period.

12. A catheter system comprising:
a catheter adapter comprising a catheter and an inlet, the catheter configured to be inserted into a patient's vasculature; and
a needle-free connector comprising a first port, a second port positioned opposite the first port, and a side port positioned between the first port and the second port, the second port comprising a valve member, wherein the needle-free connector comprises chlorhexidine.

13. The system of claim 12, wherein the chlorhexidine comprises at least one of chlorhexidine diacetate and chlorhexidine gluconate.

14. The system of claim 12, wherein the needle-free connector comprises a lube comprising the chlorhexidine.

15. The system of claim 12, wherein the needle-free connector comprises a coating comprising the chlorhexidine.

16. The system of claim 12, wherein the needle-free connector comprises an insert received within the needle-free connector, the insert comprising the chlorhexidine.

17. The system of claim 12, wherein the needle-free connector comprises a flow diverting feature, the flow diverting feature comprising an insert or coating comprising the chlorhexidine.
